(19) United States
(12) Patent Application Publication
(10) Pub. No.: US 2002/0127211 A1
(43) Pub. Date: Sep. 12, 2002

(54) ABSORPTION OF MINERALS BY INTESTINAL CELLS

(76) Inventors: DOMINIQUE BRASSART, SAINT BERTHEVIN (FR); ELISABETH VEY, GLAND (CH)

Correspondence Address: BELL, BOYD & LLOYD LLC, P. O. BOX 1135, CHICAGO, IL, 60690-1135, US

(21) Appl. No.: 09/445,796

(22) PCT Filed: Jun. 26, 1998
(86) PCT No.: PCT/EP98/04036

(30) Foreign Application Priority Data
Jul. 5, 1997 (EP) 97111380.8

(51) Int. Cl. A01N 63/00; A61K 33/24

(52) U.S. Cl. 424/93.45; 424/617

(57) ABSTRACT

A method for increasing or facilitating the absorption of minerals from the diet. A nutritional composition which contains lactobacilli is enterally administered to a mammal. The nutritional composition is suitable for the treatment or prophylaxis of sutjects having mineral deficiencies, or to compensate for physiological deficiencies due to a diet low in minerals, or to satisfy major physiological requirements for minerals in young children. pregnant women, women who are breastieeding, and the elderly.

This invention relates to a method for facilitating or increasing the absorption. by mammals, of minerals from the general diet. In particular. this invention relates to a method which involves the administration of an enteral composition containing Lactobacilli micro-oroanisms.

Minerals are kev elements in major physiological processes. Calcium is, for example. of vital importance fbr the formation of bones and teeth, muscle contraction and the synthesis of hormones. Calcium is also an essential secondary messenger in most cell activation phenomena.

Minerals. of which the diet is the primarv source. are assimilated bv the body bv crossing the intestinal mucosa so as to then pass into the blood stream. The degree of assimilation (or of absorption) of minerals by the body in fact depends both on their solubilitv in the intestinal medium and on the capacity of the intestinal cells to assimilate them and to transfer them into the blood stream (R. Wasserman et al., In Mineral Absorption in the Monogastric GI Trac. Advances in Experimental Medicine and Biology, 249. 45-65. Plenum Press, N.Y., 1989).

The location, the efficiency and the mechanisms of calcium absorption all along the intestine have been studied in rats and chickens for manv vears (Bronner F.. J. Nutr.. 122, 641-643, 1992: Schachter D., Am. J. Physiol.. 196, 357-362. 1959). For obvious ethical and technical reasons. such studies have been limited in man (Hylander E. et al.. Scand. J. Gastroenterol.. 25, 705. 1990) and only a few in vitro studies have been undertaken (Elsherydah A. et al.. Gastroenterology, 109, 876, 1995; Feher J.J.. Am. J. Physiol., 244, C303. 1983: Feher J.J., Cell Calcium, 10, 189, 1989).

One of the most widelv studied aspects of mineral absorption is the bioavailabilitv of the minerals depending on the composition of the dailv diet (Bronner F.. J. Nutr., 123, 797, 1993). However. many minerals which are highiv bioavailable are also instable and are unsuitable for use in the diet. Further, merely supplementing the diet with greater amounts of minerals often has a negative effect on the organo-leptic properties of the diet.

A possible solution to the problem is to facilitate or improve the absorption of minerals from the diet. However there have been few studies on methods of facilitating or increasing the absorption of minerals from the diet and the results have not been consistent.

Rasic et al. have reported that the minerals contained in dairy products are assimilated better when these products are fermented. This effect is attributed to the presence of acids in the fermented dairy products (XP00205223 8: In Fermented Fresh Milk Product, volume 1, p1 14-115, 1978).

More recentlv. Yaeshima et al. have also shown an increase in the absorption of calcium in rats from a diet of calcium-fortified whey when a combination of oli-osaccharides and Bifidobacteria is consumed (XP002052237: Bulletin of the International Dairy Fermentation. No. 313. 1996).

However. Kot et al. Have reported that Lactobacillus acidophilzis naturally internalizes Fe²⁺and oxidizes it to Fe³: which is an insoluble form which is more difficult to assimilate (J. Aoric. Food Chem., 43, 1276-1282, 1995).

Therefore there remains a need for a means of facilitating or increasing the absorption of minerals present in the diet.

Accordingly. this invention provides a method for increasing absorption of minerals from the diet. the method comprising enterally administering to a mammal a nutritional composition which contains a lactobacilli bacteria.

It has been surprisingly found. by use of an in vitro model. that lactobacilli are able to directlv facilitate or improve the absorption of minerals. especially calcium. bv human intestinal cells. Without wishing to be bound bv theory, this is thought to be linked to induction of acidification of the microenvironment around the intestinal cells and the bacteria in contact with the intestinal cells. Both the bacteria and the intestinal cells may participate in the induction of acidification. This localized acidification might thus play an active role in the solubilization of minerals. and therefore in the capacity of the body to assimilate them.

In another aspect, this invention provides the use of lactobacilli in the preparation of an enteral nutritional composition for facilitating or improving the absorption of minerals by the mammal. The enteral nutritional composition mav be used for the treatment or prophylaxis of mineral deficiencies

Embodiments of the invention are now described, by wav of example only, with reference to the drawings in which:

FIG. I represents the basal absorption of calcium by Caco-2 intestinal cells in the absence of lactobacilli;

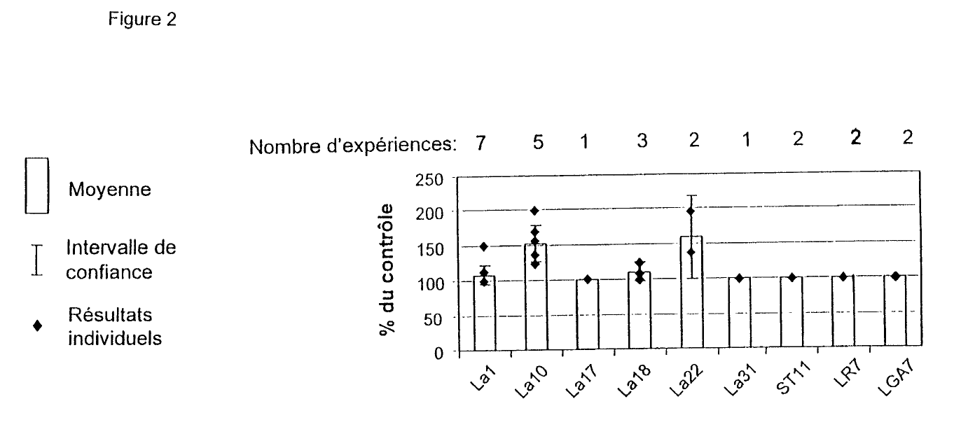

FIG. 2 represents the influence of about 6.7×10⁷ cfu/ml of various strains of lactobacilli on the absorption of calcium bv Caco-2 intestinal cells:

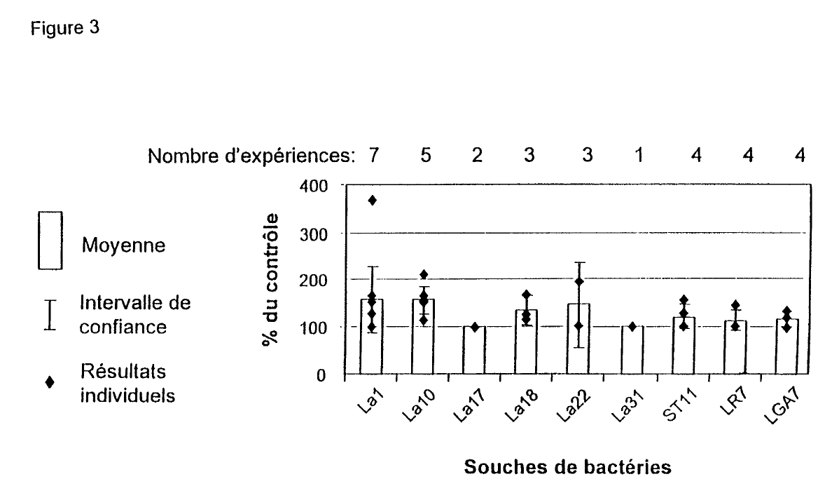

FIG. 3 represents the influence of about 3.4×10⁸ cfu/ml of various strains of lactobacilli on the absorption of calcium by Caco-2 intestinal cells.

The invention relates to the enteral administration of a nutritional composition which contains lactobacilli to facilitate or improve the absorption of minerals present in a daily diet. Examples of minerals are calcium. magnesium. iron and/or zinc. The ingestion of lactobacilli increases the bioavailabilitv of the minerals, that is to say makes the minerals. Which are often not very soluble in the intestine. more accessible to the intestinal cells.

Any food-grade. lactobacilli strain which may be used. For example, the following lactobacilli may be used: Lactobacillus acidophilis, Lactobacillus crispatits, Lactobacillis amylovorois, Lactobacillus gallinarum, Lactobacilllus gasseri and Lactobacillzs johlnsonii,- Lactobacillus paracasei: Lactobacllils reziterii;- Lactobacilluts brevis: Lactobacillus fermentum: Lactobacillzis plantartm; Lacto bacillus case i especially L. case i stbsp. casei and L. casei suzbsp. rhamnoszus: Lactobacillzls delbruckii especially L. delbruckii subsp. lactis, L. delbrtickii szubsp. helveticuts and L. delbruckii szubsp. bztlgaricis, and Leuiconostoc mesenteroides especially L. mesenteroides sztbsp. cremoris, for example (Bergeny's Manual of Systematic Bacteriology, vol. 2, 1986; Fujisawa et al.. Int. Syst. Bact. 42, 487-491. 1992).

The lactobacilli may be capable of adhering to intestinal cells but need not be. However, the lactobacilli are preferably such that at least 50 bacteria. in particular at least 80 bacteria. are capable of adhering in vitro to 100 intestinal cells. To select such an adherent type of bacteria. a culture of bacteria may be spread on a confluent culture of an immortalized line of epithelial cells of the intestine (EP 0802257). the confluent culture wNashed, and the number of bacteria adhering to the villosities of the line measured.

Probiotic lactobacilli are of particular interest. Some strains are in fact capable of adhering to human intestinal cells, of excluding pathogenic bacteria which are on human intestinal cells, and/or of acting on the human immune system by allowing it to react more strongly to external aggression (immunomodulation capacity), for example bv increasing the phagocytosis capacity of the granulocytes derived from human blood (J. of Dairy Science, 78, 491-197, 1995: immunomodulation capacitv of the La-1 strain which was deposited by Nestec SA with the treaty of Budapest in the Collection Nationale de Culture de Microorganisme (CNCM), 25 rue docteur Roux. 75724 Paris, June 30, 1992, where it was attributed the deposit number CNCM I-1225). This strain is described in EP 0577904

By way of example. it is possible to use the probiotic strain Lactobacillzis acidophilzis CNCM I-1225. This strain was recently reclassified among the Lactobacillzs johnsonii bacteria, subsequent to the new taxonomv. proposed by Fujisawa et al.. which is nowv authoritative in the field of taxonomv of acidophilic lactobacilli (Int. J. Syst. Bact., 42, 487-791, 1992). Other probiotic bacteria are also available, such as those described in EP0199535 (Gorbach etal.). U.S. Pat. No. 5296221 (Mitsuoka et al.), U.S. Pat. No. 556785 (Institut Pasteur) or US5591428 (Probi AB), for example.

The nutritional compositions preferably comprise a sufficient quantity of live lactobacilli for a facilitated absorption of minerals by the intestinal cells, for example at least 106 cfu/ml. in particular 10⁷-10¹¹ cfulml, preferably 10⁸-10¹¹cfu/ml (“cfu” means “colony forming unit”).

The nutritional composition may also contain other bacteria as desired, for example other probiotic bacteria.

The nutritional composition may also include a suitable protein source; for example an animal or plant protein source. Suitable protein sources are milk proteins soy proteins. rice proteins. wheat proteins, sorghum proteins. and the like. The proteins mav be in intact or hydrolyzed form.

The nutritional composition may also include a suitable carbohydrate source; for example sucrose. fructose. glucose. maltodextrin. and the like.

The nutritional composition mav also include a suitable lipid source; for example a suitable animal or plant lipid source. Suitable lipid sources include milk fats. sunflower oil, rapeseed oil, olive oil. safflowver oil. and the like.

The nutritional composition may also be fortified with minerals and vitamins. It is especially preferred to fortify the nutritional composition with calcium.

The nutritional compositions may be prepared in the form of food compositions intended for human or animal consumption. Suitable food compositions may be provided in the form of liquids. powders, and solids.

The nutritional composition may be fermented to obtain a sufficient quantity of lactobacilli. Fermented compositions based on milk are thus particularly suitable. The term milk applies not onlv to animal milks but also to what is commonly called a vegetable milk. that is to sav an extract of treated or untreated plant materials such as legumes (soya. chick pea. lentil and the like) or oilseeds (rape, soya. sesame. cotton and the like), which extract contains proteins in solution or in colloidal suspension, which are coagulable by chemical action. by acid fermentation and/or bv heat. It has been possible to subject these vegetable milks to heat treatments similar to those for animal milks. It has also been possible to subject them to treatments which are specific to them, such as decolorization. deodorization. and treatments for suppressing undesirable tastes.

Finallvy the word milk also designates mixtures of animal milks and of plant milks.

It is also possible to add, mix or coat the nutritional composition. during its preparation, with an appropriate quantity of a culture of lactobacilli in liquid. concentrated, drv or encapsulated form, according to need.

It has been found that the microencapsulation of the lactobacilli has therapeutic advantages. First, microencapsulation significantly increases the survival of the lactobacilli and therefore the number of live lactobacilli which arrive in the intestine. Even more importantly. the lactobacilli are gradually released into the intestine, which permits prolonged action of the lactobacilli on the absorption of minerals by the intestinal cells.

Preferably, to encapsulate lactobacilli. the lactobacilli are freeze-dried or spray-dried (EP08 18529), and they are incorporated into a gel consisting, for example. of a solidified fattv acid. a sodium alginate. polymerized hvdroxvpropylmethvlcellulose or polymerized polvvinylpyrrolidone. To this effect. the teaching given in FR2.443.247 is incorporated bv reference.

The nutritional compositions need not contain carbohydrates necessary for active fermentation bv lactobacilli in the intestinal medium. On the contrary. the facilitated absorption of minerals is independent of the fermentative activity of the lactobacilli, but rather appears to result from the direct contact between the lactobacilli and the intestinal cells. This is thought to induce acidification of the microenvironment and therefore a better solubilization of the minerals.

However, it may be desirable to provide for renewal or specific multiplication of the lactobacilli in the intestinal medium so as to prolong the effect of facilitated absorption of the minerals. This may be achieved bv adding fibres which facilitate the specific multiplication of lactobacilli in the intestinal medium to the nutritional composition. These fibres are soluble and fermentable.

These fibres mav be selected from, for example, plant pectins. chito-, fructo-. gentio-, galacto-, isomalto-, manno- or xvlo-oligosaccharides or oligosaccharides from soya. for example (Plavne et al., Bulletin of the IDF 313.

Group B42, Annual Session of September 95, Vienna).

The preferred pectins are polvmers of α- 1,4-D-galacturonic acid having a molecular weight of the order of 10 to 400 k-Da, which can be purified from carrots or tomatoes, for example (JP60164432). The preferred galacto- oligosaccharides comprise a saccharide portion consisting of 2 to 5 repeating units of structure [-α-D-Glu-(1→4)-β-D-Gal-(I<6)-] (Yakult Honsa Co.., Japan).

The preferred fructo-oligosaccharides are inulin-oligofructoses extracted from chicory which may comprise. for example. 1-9 repeating units of structure [-β-D-Fru-(1→2)-β-D-Fru-(1→2)-] (WO94/12541; Raffinerie Tirlemontoise S.A., Belgium). or olilosaccharides synthesized from sucrose units which may comprise. for example. a saccharide portion consisting of 2 to 9 repeatin2 units of structure [-α-D-Glu-(1→2)-β-D-Fru-(19)-] (Meiji Seika Kasiha Co., Japan).

The preferred malto-oligosaccharides comprise a saccharide portion consisting of 2 to 7 repeating units of structure [-α-D-Gal-(1→4)-] (Nihon Shokuhin Kako Co., Japan). The preferred isomaltoses comprise a saccharide portion consisting of 2 to 6 repeating units of structure [-α-D-Glu-(1→6)-] (Showa Sangyo Co., Japan). The preferred gentio-oliaosaccharides comprise a saccharide portion consisting of 2 to 5 repeating units of structure [-β-D-Glu-(16)-] (Nihon Shokuhin Kako Co.. Japan). Finally. the preferred xvlo-oligosaccharides comprise a saccharide portion consisting of 2 to 9 repeating units of structure [-Fe-x,yl -(1→4)-] (Suntory Co.. Japan). for example.

The quantity of fibres in the nutritional composition depends on their capacity to promote the development of lactobacilli. As a general rule, the nutritional composition may contain from 1 to 50% of such fibres (by weight relative to the dry matter). The concentration of lactobacilli may be at least 10³ CFU of lactobacilli per g of fibres, preferably 104 to 10⁷ CFU/g of fibres.

Another advantage provided by the fibres consists in the fact that the intestinal transit is retarded bv the fibres. This is particularly the case if the quantitv of fibres is large, that is to say of the order of 20-50% relative to the weight of the composition. The lactobacilli being gradually eliminated by the action of the intestinal transit. it is possible, in this manner. to prolong the beneficial action of the lactobacilli on the absorption of minerals by the intestine.

The nutritional compositions may be in the form of any suitable enterally administered food. For example, the nutritional composition may take the form of a fermented milk (EP0577904), an infant (EP0827697). a fromage frais (PCT/EP97/06947). a ripened cheese. an ice cream (WO 98/09535), a biscuit filled with a cream (EP704164; EP66603 1), a dry sausage and/or a pate (EP689769).

The nutritional compositions may also be in a form suitable for people who cannot tolerate dairy products. These nutritional compositions will not contain allergenic milk derivatives. For example. for children Who are allergic to milk proteins, the nutritional composition may be formulated to contain hypoallergenic milk derivatives. These milk derivatives may be in accordance with European directive 96/4/EC which states that in a hypoallergenic milk. the allergenic proteins should be immunologically at least 100 times less detectable than in a nonhydrolysed milk (Off. J. Europ. Comm.. NoL49/12. annex point 5.a. 1996: Fritsche et al.. Int. Arch. Aller. and Appl. lmm.. 93, 289-293, 1990).

The nutritional compositions are particulariv suitable for the treatment or prophylaxis of people having mineral deficiencies. or to compensate for physiological deficiencies due to a diet loxv in minerals, or to satisfy major physiological requirements for minerals in children. pregnant women. women who are breastfeeding and the elderly.

This invention is now further described by means of specific examples. The percentages are given by xveiht unless otherxvise indicated. These examples are given by way of illustration only and do not in anv manner constitute a limitation of the invention.

### Example 1

Materials: ⁴⁵CaCl, is obtained from Amersham. Lucifer yellow from Si2ma. collagen I from Centrix Pharmaceuticals, PBS, HEPES and the components of the cell culture medium from Gibco, and the culture supports from Falcon.

Cell culture: the human cell line Caco-⁹, isolated from a colon adenocarcinoma. is obtained from American Type Culture Collection (passage 41). The cells are placed in culture in an amount of 4×1I⁴ cells/cm² in DMEM containing 4.5 g/l of glucose. 20% heat-inactivated foetal calf serum, 1 mg/ml of fungizone, 100 U/ml of penicillin/streptomycin. 200 pg/ml of gentamycin and 1% of nonessential amino acids. The cells are regularly tripsinized and placed in culture aaain at 1:20. The cells used in the calcium transport experiments are placed in culture at 1×10⁵ cells/cm² in permeable inserts previously coated with a layer of collagen I at 50 pLg/ml. In all cases. the cells are maintained in a 10% CO₂/90% air incubator at 37° C. and the medium is replaced every two days.

Viabilitv of the Caco-2 cells: in order to exclude the possibility that the potentiation of the absorption of calcium by the intestinal cells in the presence of lactobacilli is due to cellular damage, a portion of each sample serving for the assay of calcium was used for an assay of the hexosaminidase activity (Landegren et al., J. Immunol. Method 67, 379-378. 1984). This calorimetric test makes it possible to quantit cell lvsis and/or death by measuring the hexosaminidase activitv released into the supernatant from the cytosol of damaged cells. The results show that in all the experiments, the hexosaminidase activitv is equivalent in the presence of lactobacilli.

Permeabilitv of the cellular lawn: the intearitv of the lawn formed bv the Caco- 2 cells at the end of their growth and oftheir differentiation is evaluated bv measuring the transepithelial electrical resistance (TEER) using a voltmeter/ohmmeter Millicell-ERS. The calcium absorption experiments are carried out when this resistance reaches at least 700 ohm×cm². The permeability of the cellular lawn during the calcium absorption experiments is evaluated b measuring the level of diffusion (in %) of Lucifer vellow, a molecule which does not cross the cell membrane.

Transport of calcium: the Caco-2 cells are cultured on inserts for 3 to 5 weeks. On the day of the experiment. the cellular lawn is washed twice in PBS and then the bottom compartment of the insert incorporating the serosa (basolateral pole of the cells) receives 1.5 ml of carrier butTer (140 mM NaCl. 5.8 mM KC[. 0.34 mM NaH PO₄ 0.44 mM KHIPO₄, 0.8 mM M2SO₄. 20 mM HEPES. 4 mM glutamine, 25 mM glucose, pH 7.4) supplemented with 2.5 mM CaCI₂. whereas the top compartment of the insert incorporating the intestinal lumen (apical pole of the cells) receives 1.5 ml of carrier buffer supplemented with 10 mM CaCl₂ and trace amounts of 4⁵CaCl₂ and Lucifer yellow. The inserts are then placed at 37° C. and 50 Kil of sample in the bottom and top compartments are removed at regular intervals.

The radioactivity contained in these samples is evaluated by liquid scintillation counting and makes it possible to extrapolate on the quantity of coicl CaCl₂ absorbed. The basal transport of calcium is expressed as nmol of calcium transported to the bottom compartment of the insert. The diffusion of Lucifer yellow detected by spectrofluorometrv in the bottom compartment is expressed in % of the quantitv introduced into the top compartment.

Influence of the lactobacilli: the strains Lactobacillus johnsonii Lal (CNCM 1-1225), La17. La22. La31Lactobacillits acidophilits LaI0, LaI8. La31

Lactobacillits bulgaricits L fiS. YL8. Lactobacillus paracasei STI 1l Lactobacillits gasseri LGA7: Lactobacillus reuteri LR7 and Streptococczws thermophiluts Sfi20, YS4 (Nestec collection, Lausanne. Switzerland) are placed in culture under anaerobic conditions in MRS broth for Lactobacillus or M1 7 for Streptococcus for two times 24 h, washed in PBS and resuspended in carrier buffer before being introduced into the top compartment of the inserts. The Caco-2:bacteria ratio is then about 1: 100 according to the tests (6.7×10⁷ or 3.4×10⁸ cfu/ml in the top compartment of the inserts. for the tests presented in FIGS. 2 and 3) The absorption of calcium is evaluated according to the protocol mentioned above.

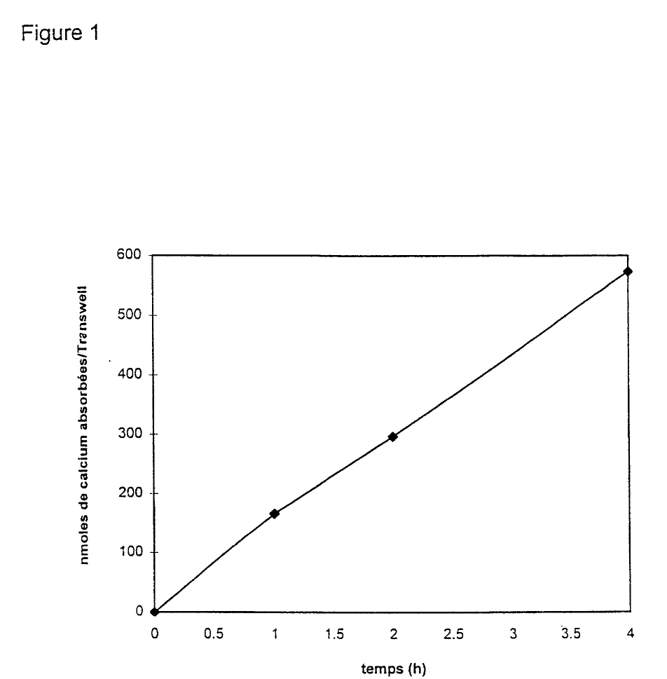

Results of the basal transport of calcium: a calcium Lyradient was established in the inserts by introducin2 2.5 mM CaCl, into the bottom compartment. which corresponds to the normal human plasma concentration, and arbitrarily 10 mM CaCl₂ into the top compartment. which would correspond to the calcium content of a food diet. As shown bN the results of a representative experiment illustrated bv FIG. 1, the basal absorption of calcium by the Caco-2 cells increases with time to reach up to 600 nmol/insert. comprising about 3×10⁶ cells. after 4 h. As a check for the intearity of the cellular lawn during the experiment, the diffusion of Lucifer y ellowv was measured and proved to be less than ²%.

Measurement of the influence of lactobacilli: in FIGS. 2 and 3. the absorption of calcium by the Caco-2 cells is increased significantly in the presence of the adherent Lactobacillzisjohnsonii strains La 1 and La22. in the presence of the non-adherent La IO and La 18 Lactobacilluts acidophilus strains, and in the presence ofthe L. paracasei (STI 1). L. gasseri (LGA7) and L. reziterii (LR7) strains.

The capacity of the bacteria to adhere to the intestinal cells therefore does not appear to correlate directlv with their capacity to increase the absorption of calcium by these same cells. In all these experiments, the diffusion of Lucifer yellow is modulated in a similar manner but remains negligible. 30 A decrease in pH in the top compartment of the inserts is also observed when the Caco-2 cells are in the presence of lactobacilli, regardless of the strain. except with the Sfi2O strain (Table 1). There is therefore no correlation between the increase in the absorption of calcium and this decrease in pH. However certain bacterial strains capable of increasing the absorption of calcium are not 35 capable of acidifving the experimental medium in the absence of Caco-2. This means that the acidification in the presence of Caco-2 and of bacteria requires a collaboration between the two types of organisms and could be due to the Caco-2 cells.

TABLE 1
Influence of lactobacilli on the pH of the experimental medium in the
absence or in the presence of Caco-2 cells

| | Number of | | |
|---|---|---|---|
| Bacteria | tests | pH without Caco-2 | pH with Caco-2 |
| None | 4 | 7 +/− 0 | 7 +/− 0 |
| La1 | 3 | 6.75 +/− 0.3 | 3.75 +/− 0.3 |
| La10 | 3 | 4.65 +/− 0.3 | 4.15 +/− 0.3 |
| La17 | 2 | 7 +/− 0 | 3.5 +/− 0.7 |
| La18 | 2 | 7 +/− 0 | 3.5 +/− 0.5 |
| La22 | 2 | 7 +/− 0 | 3.25 +/− 0.35 |
| La29 | 2 | 4.25 +/− 0.35 | 3.5 +/− 0 |
| La31 | 2 | 7 +/− 0 | 3.75 +/− 0.35 |
| Sfi20 | 1 | 7 | 7 |
| YS4 | 1 | 5 | 4 |
| Lfi5 | 1 | 4 | 3 |
| YL8 | 1 | 4 | 3 |

### Example 2

Tests similar to those carried out in Example I were carried out to determine the influence of lactobacilli on the absorption of calcium by the intestinal cells in the presence of labelled inulin (³H-inulin, Amersham- tracer prebiotic fibre). The results confirm that lactobacilli increase in vitro the absorption of minerals bv the intestinal cells.

### Example 3

Tests similar to those carried out in Example I were carried out in order to determine the influence of lactobacilli on the absorption of magnesium, iron and zinc by the intestinal cells. The results confirm that lactobacilli increase in vitro the absorption of minerals by the intestinal cells.

### Example 4 Encapsulation of lactic acid bacteria

In a 100 I tank, 80 1 of culture medium having the following composition, in % are prepared:

| | |
|---|---|
| Yeast extract | 0.25% |
| Trypticase | 1.00% |
| Phytone | 0.50% |
| Glucose | 1.50% |
| L-cysteine HCl | 0.05% |
| K₂HPO₄ | 0.25% |
| ZnSO₄ | 0.025% |
| FeCl₃ | Trace |
| Water | Balance to 100% |

Inoculation is carried out with 11 of a 20 h culture of Lactobacillus johnsonii Lal (CNCM I-1225). The medium is incubated for 12 h at 30° C. The culture broth is centrifuged and 240 g of cells are recovered. They are diluted in 250 ml of skimmed milk supplemented with 7% lactose. The mixture is frozen using liquid nitrogen. The freeze-drying is performed at 40° C. overnioht. A 5% dispersion of the powder obtained is prepared in hydrogenated vegetable fat having a melting point of 42° C. and liquefied at 45° C. The dispersion is injected at 45° C. under a pressure of 4 bar, at the same time as liquid nitrogen, in an amount of I part of dispersion for 5 parts of nitrogen, at the top of a vertical cylinder 1.5 m in diameter and 10 m high. A container is placed at the bottom of the cylinder. which contains liquid nitrogen in which the microbeads containing the bacteria whose diameter varies between 0.1 and 0.5 m are collected. The microbeads are then placed in a fluidized bed and an alcoholic solution containing 8% zein is sprayed over the bed, in a quantity such that the zein layer formed around the microbeads represents 5% of their weight.

The microbeads are then incorporated into a food composition intended to facilitate the absorption of minerals by the intestinal cells.

### Example 5

A concentrated base for ice cream is prepared by mixing at 60-65° C. for 20 min about 11% of lactic fat, 8.8% of milk solids (solids-not-fat). 25% sucrose. 5% of glucose syrup and 0.6% of Emulstab ⁰ SE3O. The base is homogenized at 72-75° C and at 210 bar (2 stages at 2 10/50 bar), it is pasteurized at 85° C. for 22 sec (APV pasteurizer, France, Evreux, 400 1/h), it is cooled to 4° C. and 40% of milk acidified by Lactobacillusjohnsonii La-] (5×10⁸ cfu/mI) and Bifidobacterium longtim Bil 6 (3×10⁸ cf)/ml) strains is added thereto. The composition of this concentrated base is presented in the table below.

| | | | | | Dry |
|---|---|---|---|---|---|
| | Composition | | Solids-not- | Sucrose | extract |
| Ingredients | (kg) | Fat (%) | fat (%) | (%) | (%) |
| Cream | 31.43 | 11.00 | 1.57 | | 12.57 |
| (35%) | | | | | |
| Skimmed | 7.60 | | 7.30 | | 7.30 |
| milk powder | | | | | |
| Sucrose | 36.77 | | | 25.00 | 25.00 |
| Glucose | 5.27 | | | | 5.00 |
| syrup | | | | | |
| Emulstab ® | 0.67 | | | | 0.63 |
| SE30 | | | | | |
| Water | 18.26 | | | | |
| Total: cream | 100.00 | 11.00 | 8.87 | 25.00 | 50.50 |
| base | | | | | |
| Cream base | 60.00 | 6.60 | 5.32 | 15.00 | 30.30 |
| (60%) | | | | | |
| Acidified | 40.00 | 1.40 | 4.68 | — | 6.08 |
| milk (40%) | | | | | |
| Total: cream | 100.00 | 8.00 | 10.00 | 15.00 | 36.38 |
| base + | | | | | |
| acidified | | | | | |
| milk | | | | | |

After maturation of the cream for 12 h at 5° C., it is frozen to an overrun of 95% by volume (Crepaco freezer, France, Evreux; 160 1 of product/h).

A wafer dough is prepared which contains 10% fructo-oliaosaccharide Raftilose® L30 (Raffinerie Tirlemontoise S.A., BE). according to the recipe reproduced in the table below. After baking. the wafer is conventionally formed into a cone. After cooling, the inside of the cones is spray-coated with a fatty film and then the cones are filled with the whipped ice cream described above. For an 11.5 g wafer cone. 130 ml of whipped ice cream (about 65 g) and 5 g of chocolate (spraying over the cream) are thus used.

| Ingredient | Weight (g) | Supplier |
|---|---|---|
| Ordinary wheat flour 55 | 52 | |
| Starch | 0.2 | |
| Fructo-oligosaccharide | 10 | Raffinerie Tirlemontoise S.A., |
| Raftilose ® L30 | | BE |
| Sugar | 27.8 | |
| Fat | 8 | |
| Emulsifier | 1.5 | |
| Salt | 0.5 | |
| Total: wafer recipe | 100 | |

1.1 g of fibres and about 108 cfu/g of lactobacilli are thus provided per ice cream cornet. The fibres, by promoting the specific development of lactobacilli in the intestinal tract. thus promote the assimilation of minerals.

1. Use of lactobacilli in the preparation of an enteral nutritional composition for facilitating or improving the absorption of minerals by a mammal.

2. Use according to claim I in which the lactobacilli is a Lactobacillus bacteria which is capable of adhering to intestinal cells.

3. Use according to claim 2 in which the lactobacilli is the Lactobacilltis johnsonii CNCM I- 1225 strain.

4. Use according to claim I in which the enterai nutritional composition contains 10⁷to 10¹¹ cfu of lactobacilli.

5. Use according to claim I in which the enteral nutritional composition facilitates the absorption of calcium. magnesium, iron and/or zinc.

6. Use according to claim I in which the enteral nutritional composition contains mlilk proteins.

7. Use according to claim 6 in which the enteral nutritional composition is an infant formula comprising hypo-allergzenic milk protein hydrolysates.

8. Use according to claim 1 in which the enteral nutritional composition further comprises prebiotic fibres.

9. Use of lactobacilli i n the preparation of an enteral nutritional composition for the treatment or prophylaxis of mineral deficiencies.

10. A method for increasing absorption of minerals from the diet the method comprising enterally administering to a mammal a nutritional composition which contains lactobacilli.
